(19) United States
(12) Patent Application Publication
(10) Pub. No.: US 2026/0224151 A1
(43) Pub. Date: Aug. 6, 2026

(54) DETECTING CARDIOVASCULAR CONDITION FROM ELECTROCARDIOGRAM DATA USING ENSEMBLE OF ARTIFICIAL INTELLIGENCE MODELS

(71) Applicant: Tempus AI, Inc., Chicago, IL (US)

(72) Inventors: Nicole Jane Rebecca Hakim, Miami, FL (US); Greg Sungyul Lee, Chicago, IL (US)

(21) Appl. No.: 19/042,728

(22) Filed: Jan. 31, 2025

(51) Int. Cl. A61B 5/346; A61B 5/00; G16H 10/60; G16H 20/00; G16H 50/20; G16H 50/30

(52) U.S. Cl. CPC A61B 5/346; A61B 5/7264; A61B 5/7275; G16H 10/60; G16H 20/00; G16H 50/20; G16H 50/30

(57) ABSTRACT

Techniques for detecting a cardiovascular condition from electrocardiogram (ECG) data using an ensemble of artificial intelligence models are disclosed. One or more demographics of a subject are obtained. ECG data of the subject is obtained. The ECG data is segmented and provided, with the one or more demographics, as input to a first trained artificial intelligence (AI) model having a first framework and a second trained AI model having a second framework different from the first framework. Cardiovascular scores are obtained from the trained AI models. A collective cardiovascular score is determined based on the cardiovascular scores. The collective cardiovascular score is compared to an operating point threshold obtained based on one or more demographics. An indication of a cardiovascular condition is determined based on the comparison.

### BACKGROUND

The cardiovascular system is critical to life and impacts every system in the body. For example, the cardiovascular and pulmonary system operate in close cooperation to oxygenate blood in the body. Thus, heart conditions often impact the pulmonary system and vice versa. Because systems of the body are directly impacted by cardiovascular function, measures of cardiovascular function can indicate conditions of these other systems.

Pulmonary hypertension (i.e., “PH”) is a condition characterized by elevated blood pressure in the lungs. Pulmonary hypertension is correlated with serious cardiovascular conditions such as heart failure. Detecting pulmonary hypertension allows subjects to receive treatment for the condition, improving subject outcomes.

### BRIEF SUMMARY

Techniques for detecting a cardiovascular condition from electrocardiogram data using an ensemble of artificial intelligence models are disclosed. One or more demographics of the subject are obtained. Electrocardiogram (ECG) data of a subject is received, wherein the ECG data includes a time series of voltage measurements. The ECG data is segmented into a plurality of ECG data windows. The plurality of ECG data windows and the one or more demographics are provided as input to a first trained artificial intelligence (AI) model having a first framework and a second trained AI model having a second framework different from the first framework. A first cardiovascular score is received via the first trained AI model and a second cardiovascular score is received via the second trained AI model. A collective cardiovascular score is determined based on the first cardiovascular score and the second cardiovascular score. An operating point threshold is identified from a plurality of operating point thresholds based on the one or more demographics. The collective cardiovascular score is compared to the operating point threshold. An indication of a cardiovascular condition is determined based on the comparison. In some embodiments, a notification is provided based on the indication of the cardiovascular condition.

### BRIEF DESCRIPTION OF THE SEVERAL VIEWS OF THE DRAWINGS

Non-limiting and non-exhaustive embodiments are described with reference to the following drawings. In the drawings, like reference numerals refer to like parts throughout the various figures unless otherwise specified.

For a better understanding, reference will be made to the following Detailed Description, which is to be read in association with the accompanying drawings:

FIG. 1A is a system diagram illustrating a system used to detect a cardiovascular condition using an ensemble of artificial intelligence models in some embodiments.

FIG. 1B is a diagram illustrating an example of electrocardiogram data segmentation in some embodiments.

FIG. 2 is a logical flow diagram illustrating a process for detecting a cardiovascular condition using an ensemble of artificial intelligence models in some embodiments.

FIG. 3 is a logical flow diagram illustrating a process for selecting training data for training an artificial intelligence model in some embodiments.

FIG. 4 is a logical flow diagram illustrating a process for setting an operating point for subjects having one or more demographics in some embodiments.

FIG. 5 is a block diagram illustrating dataset creation for training an artificial intelligence model in some embodiments.

FIG. 6 is a block diagram illustrating a first artificial intelligence model architecture in some embodiments.

FIG. 7 is a block diagram illustrating a second artificial intelligence model architecture in some embodiments.

FIG. 8 is a block diagram illustrating a third artificial intelligence model architecture in some embodiments.

FIG. 9 is a block diagram illustrating a fourth artificial intelligence model architecture in some embodiments.

FIG. 10 shows a system diagram that describes one implementation of computing systems for implementing embodiments described herein.

### DETAILED DESCRIPTION

The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks and the automobile environment, have not been shown or described in order to avoid unnecessarily obscuring descriptions of the embodiments. Additionally, the various embodiments may be methods, systems, media, or devices. Accordingly, the various embodiments may be entirely hardware embodiments, entirely software embodiments, or embodiments combining software and hardware aspects.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described, unless the context clearly dictates otherwise. In addition, throughout the specification, the meaning of “a,” “an,” and “the” include singular and plural references.

Cardiovascular disease is the leading cause of mortality in the United States and worldwide. Pulmonary hypertension (i.e., “PH”) is correlated with serious cardiovascular conditions. In various cases, PH is caused by heart failure, diseases, etc. PH can also exacerbate or cause conditions such as heart failure.

Pulmonary hypertension is typically diagnosed using a transthoracic echocardiogram (i.e., “echo,” “echocardiogram,” or “TTE”) or right heart catheterization (i.e., “RHC”). But these tests are relatively expensive, requiring specialized training to administer. Right heart catheterization is also an invasive procedure, involving inserting a catheter into the cardiovascular system to measure pressure in the lungs. As a result, subjects are unlikely to have echo or RHC test results available unless PH is already suspected. Additionally, the symptomatology of PH is often nonspecific, including symptoms such as shortness of breath, fatigue, dizziness, etc. Accordingly, even symptomatic subjects may not initially be tested for PH. These factors may lead to underdiagnosis of PH, preventing subjects from receiving appropriate treatment for the condition. However, electrocardiograms (i.e., “ECGs”) are relatively inexpensive to administer and are often performed as part of routine checkups. Thus, ECG data is often available for subjects. The inventors have recognized that ensemble AI models according to embodiments described herein can use ECG data to predict a variety of cardiovascular conditions such as low ejection fraction, coronary artery disease, peripheral artery disease, carotid artery disease, heart failure, arrhythmia, stenosis, regurgitation, congenital heart disease, etc. Embodiments described herein can also use ECG data to predict pulmonary conditions such as chronic obstructive pulmonary disease (i.e., “COPD”), pulmonary fibrosis, pulmonary embolism, asthma, pulmonary hypertension, etc. Various embodiments described herein are used to predict any condition indicated by ECG data.

As used herein, the term “framework” refers to an AI model architecture such as Inception or ResNet, a training dataset, an objective function, a hyperparameter, a training process, etc., or any combination thereof used to produce a trained AI model. In one non-limiting example, a first framework for an AI model includes a ResNet architecture, a selected training dataset, and a process for training the AI model using the selected training dataset. In another non-limiting example, a second framework for an AI model includes an Inception architecture. The term “framework” also includes a performance characteristic of the trained AI model, such as a type of feature extracted by the trained AI model. In one non-limiting example, a third framework for an AI model includes that the AI model extracts hierarchical features from time-series data.

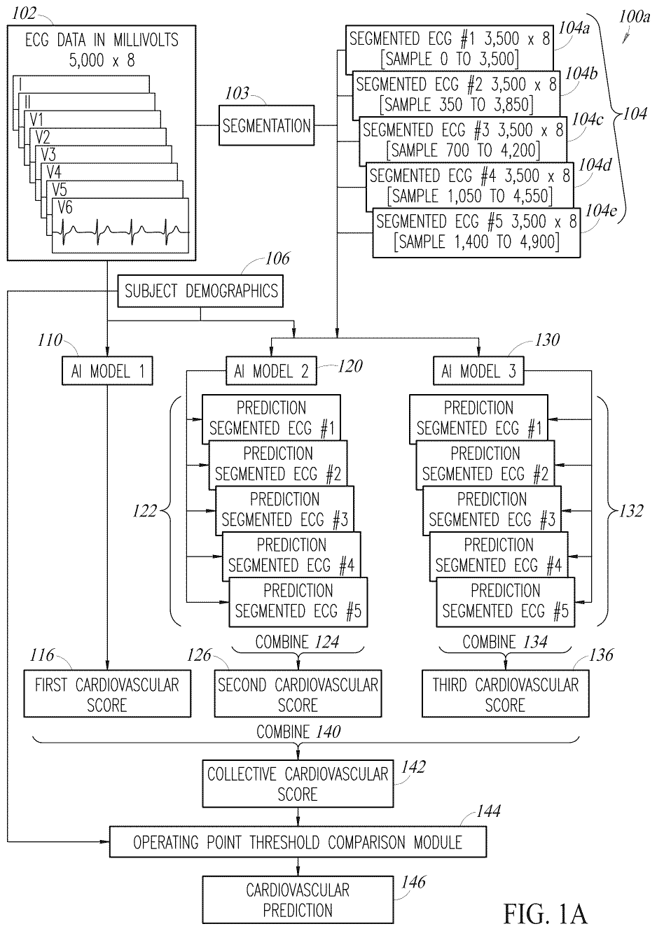

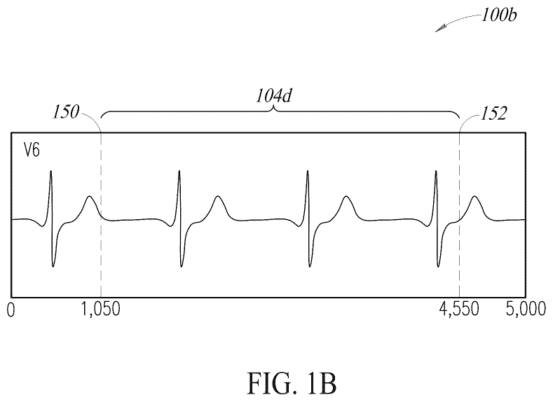

FIG. 1A is a data flow diagram illustrating data flow in a system 100a used to detect a cardiovascular condition using an ensemble of artificial intelligence models in some embodiments.

System 100a includes an ensemble model including first AI model 110, second AI model 120, and third AI model 130. ECG data 102 is segmented into segmented ECG data 104 using segmentation module 103. Segmented ECG data 104 is provided to second AI model 120 and third AI model 130, while ECG data 102 is provided to first AI model 110. In some embodiments, subject demographics 106 are provided to each of the AI models. First AI model 110 produces first cardiovascular score 116 based on ECG data 102 and subject demographics 106. Second AI model 120 and third AI model 130 generate predictions 122 and predictions 132, respectively, which correspond to segmented ECG data 104. Combination module 124 combines predictions 122 into second cardiovascular score 126. Combination module 134 combines predictions 132 into third cardiovascular score 136. First cardiovascular score 116, second cardiovascular score 126, and third cardiovascular score 136 are combined into collective cardiovascular score 142 using combine 140. Operating point threshold comparison module 144 determines, using subject demographics 106, an operating point to compare to collective cardiovascular score 142. Cardiovascular prediction 146 is based on the comparison of the collective cardiovascular score 142 to the operating point.

ECG data 102 includes time-series voltage data from an ECG test of a subject. In some embodiments, the ECG data includes data collected from a 12-lead ECG test. 12-lead ECG tests typically produce 12 sets of timeseries data corresponding to each of 12 leads contacting a subject during the test. During a 12-lead ECG test, leads V1, V2, V3, V4, V5, and V6 contact the subject's chest, while leads I, II, III, IV, V, and VI contact the subject's limbs. As shown in FIG. 1A, ECG data 102 includes timeseries data corresponding to limb leads I and II, and chest leads V1, V2, V3, V4, V5, and V6. But the disclosure is not so limited. In various embodiments, any combination of timeseries data corresponding to chest leads, limb leads, or any combination thereof, is used. Additionally, data obtained using any non-standard configuration of ECG leads may be used. In some embodiments, the non-standard configuration of ECG leads includes using fewer leads, such as by using an accessory as described in U.S. application Ser. No. 17/814,229, filed Jul. 21, 2022, and entitled “TRANSLATING AI ALGORITHMS FROM 12-LEAD CLINICAL ECGS TO PORTABLE AND CONSUMER ECGS WITH FEWER LEADS,” which is hereby incorporated by reference in its entirety. While FIG. 1A indicates that 5,000 datapoints are included for each of 8 timeseries, the number of datapoints in ECG data 102 may vary based on a sample rate of the ECG machine used to measure the ECG data or other factors.

In various embodiments, ECG data 102 includes ECG data captured over any range of time such as 1 second, 5 seconds, one minute, one hour, one day, etc. In one non-limiting example, ECG data 102 includes ECG data captured over several days using a Holter monitor.

In various embodiments, ECG data window 102 includes one or more intervals based on one or more heartbeats, ECG QRS parameters, or other measurement. In one non-limiting example, ECG data window 102 includes a plurality of intervals corresponding to a plurality of heartbeats.

In various embodiments, a length of ECG data 102 is based on a time interval, a sampling frequency of an ECG machine used to obtain ECG data 102, or both. In some embodiments, the sampling frequency of the ECG machine is between 50 Hz and 50,000 Hz. In some embodiments where the sampling frequency exceeds a configurable threshold, ECG data 102 is downsampled. In some embodiments, ECG data 102 is downsampled using a fast Fourier transform, a wavelet transform, decimation, etc. In some embodiments, ECG data 102 is downsampled using an artificial intelligence model such as a machine learning model, neural network, large language model (i.e., “LLM”), etc.

Segmentation module 103 is configured to segment ECG data 102 into segmented ECG data 104, which includes ECG data segments (i.e., “ECG data windows”). In various embodiments, segmentation module 103 segments ECG data 102 according to a window size that determines a size of each ECG data segment and a step size that determines a number of samples between the start sample of two consecutive windows. In the example shown in FIG. 1B, the window size is 3,500 samples and the step size is 350. Accordingly, first ECG data segment 104a includes samples 0 to 3,500, second ECG data segment 104b includes samples 350 to 3850, third ECG data segment 104c includes samples 700 to 4,200, etc.

In some embodiments, the step size, window size, or both, are determined based on a characteristic of ECG data 102. In some embodiments, the window size, step size, or both, are determined such that one or more ECG data segments include ECG data associated with a selected number of heartbeats. In one non-limiting example, the window size and step size are determined such that each ECG data window includes ECG data that corresponds to exactly one heartbeat.

In some embodiments, the step size, window size, or both, vary between ECG data windows. For example, when the subject's heart rate varies over a time period included in ECG data 102, the step size may vary such that each ECG window includes ECG data associated with exactly one heartbeat.

In various embodiments, segmentation module 103 segments ECG data 102 based on a number of datapoints included in ECG data 102. In some embodiments, segmentation module 103 segments ECG data 102 so that the ECG windows include a target number of datapoints or a target duration of ECG data window 102. In one non-limiting example, ECG data 102 has a duration of 10 seconds and a sampling rate of 250 HZ. Accordingly, ECG data 102 includes 2,500 datapoints. When the target duration is 3 seconds, ECG data 102 is segmented such that each ECG window includes 750 datapoints.

Subject demographics 106 include one or more demographics of the subject. In various embodiments, the one or more demographics of the subject include age, sex, hospital setting in which the ECG data was collected, body mass index (i.e., “BMI”), height, weight, smoking or other substance use status, institution at which the ECG data was collected, history of conduction disorder, history of coronary re-vascularization, history of diabetes, history of myocardial infarction, ECG machine manufacturer, ECG machine model, etc., or any combination thereof.

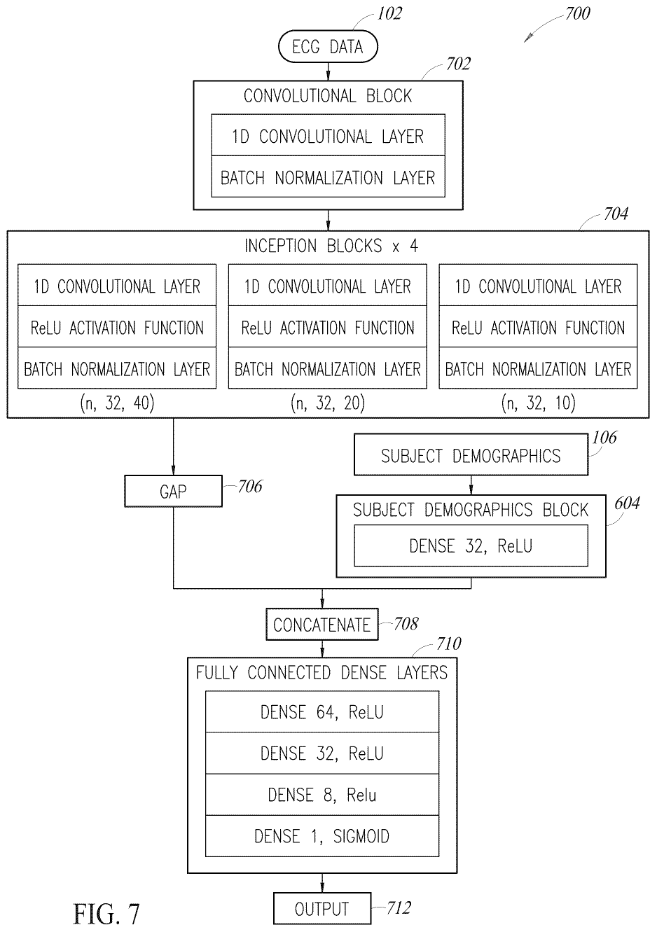

First AI model 110 is configured to generate first cardiovascular score 116 based on ECG data 102 and subject demographics 106. In some embodiments, first AI model 110 is based on an inception architecture, as discussed with respect to FIG. 7.

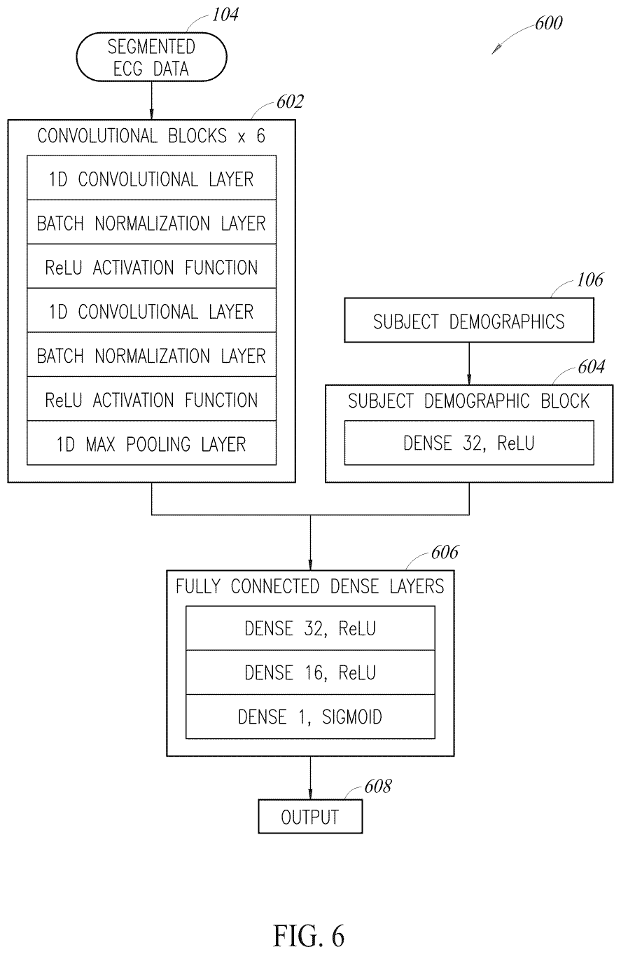

Second AI model 120 is configured to generate second cardiovascular score 126 based on segmented ECG data 104 and subject demographics 106. In some embodiments, second AI model 120 is based on a base convolutional neural network (CNN) architecture, as discussed with respect to FIG. 6.

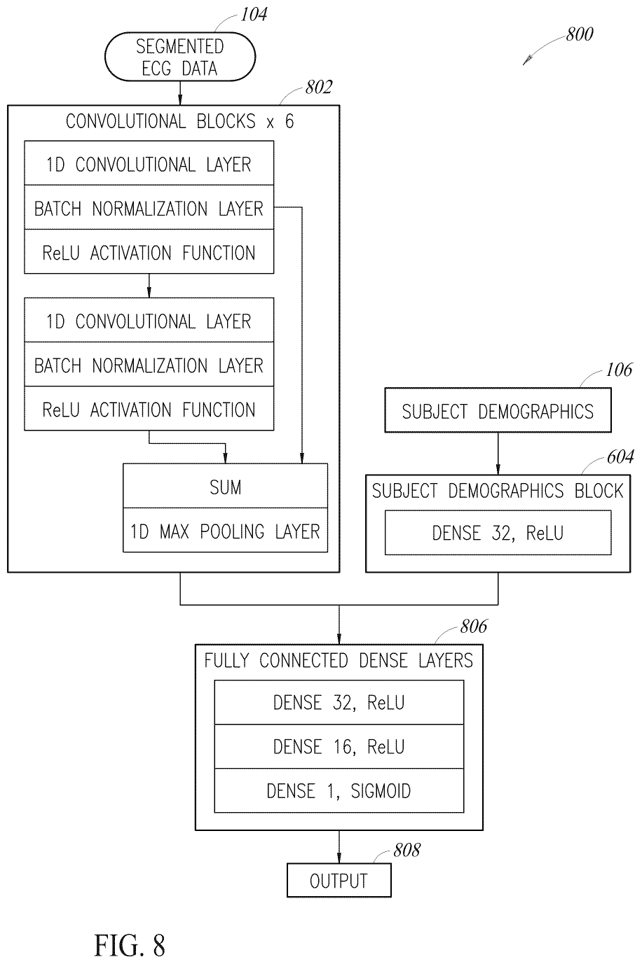

Third AI model 130 is configured to generate third cardiovascular score 136 based on segmented ECG data 104 and subject demographics 106. In some embodiments, third AI model 130 is based on a ResNet architecture, as discussed with respect to FIG. 8.

While the AI model ensemble is depicted as including three AI models in FIG. 1A, the disclosure is not so limited. In various embodiments, any number of two or more AI models is used. In some embodiments, two or more of the AI models have a same framework. In some embodiments, each AI model has a different framework.

While for ease of discussion AI models 110, 120, and 130 are at times described as based on an inception architecture, a base CNN architecture, and a ResNet architecture, respectively, the disclosure is not so limited. In various embodiments, the AI models are based on any CNN architecture such as base CNN, Inception, MobileNet, VGGNet, etc. In various embodiments, the AI models are based on any architecture capable of operating on time-series data such as long short-term memory network (“LSTM”), recurrent neural network (“RNN”), gated recurrent unit (“GRU”), transformer, etc.

In some embodiments, one or more AI models are selected to extract a particular type of feature from the ECG data or provide another performance feature to the AI ensemble model. In one non-limiting example, an AI model based on the Inception architecture is selected to extract multiscale data from the ECG data. In one non-limiting example, an AI model based on a base CNN architecture is selected to reduce overfitting. In various embodiments, multiple AI models having different feature extraction characteristics are selected to increase a variety of features extracted from the ECG data by which to predict a cardiovascular condition.

The inventors have recognized that including several AI models having different frameworks in system 100a improves overall performance of the ensemble model in predicting cardiovascular or other indicated conditions using ECG data. As discussed with respect to FIGS. 6, 7, and 8, various AI model architectures have different performance characteristics that vary the type of features extracted by the AI model from the input ECG data. For example, an AI model based on the Inception architecture described with respect to FIG. 7 may be configured to extract both local and global features of the ECG data, potentially enabling the ensemble model to identify relationships between the ECG data and a cardiovascular condition on a relatively large timescale, such as over the course of several heartbeats. An AI model based on the ResNet architecture described with respect to FIG. 8 may be configured to increase non-linearity of the ensemble model or extract complex features from the ECG data. By including two or more models having different architectures or performance characteristics in the ensemble model, the ensemble model can access a greater variety or number of features from the ECG data. The inventors have determined that ensemble models including AI models based on two or more architectures typically exhibit improved performance compared to ensemble models including AI models based on a single architecture. For example, the inventors have determined that an ensemble model including a first AI model based on an Inception architecture, a second AI model based on a ResNet architecture, and a third AI model based on a base CNN architecture provides improved performance at reduced computational cost over other combinations of AI models such as an ensemble of ten base CNN models.

Table 1 includes testing data demonstrating performance of ensemble models using various combinations of AI models on a task to predict whether a subject has pulmonary hypertension based on ECG data. Each column includes performance data corresponding to a different ensemble model.

TABLE 1

| | | 1 model | 1 model | 1 model | 1 model ResNet | 2 models | |
|---|---|---|---|---|---|---|---|
| | | (seed 2; 5s) | (seed 64000; 7s) | AF arch | arch | (5s) | |
| # models | — | 1 | 1 | 1 | 1 | 2 | |
| AUC-ROC | SaMD 40+ | 0.8278 | 0.8298 | 0.8312 | 0.8293 | 0.8332 | |
| | [90-0 days] | [0.8186, 0.8367] | [0.8207, 0.8384] | [0.8257, 0.8367] | [0.8175, 0.8405] | [0.8258, 0.8403] | |
| | SaMD 18+ | 0.8458 | 0.8495 | 0.8511 | 0.8463 | 0.8510 | |
| | [30-30 days] | [0.8331, 0.8577] | [0.8395, 0.8589] | [0.8426, 0.8591] | [0.8211, 0.8686] | [0.8403, 0.8612] | |
| | 2002-2020 | | | | | | |
| | SaMD 18+ | 0.8413 | 0.8433 | 0.8444 | 0.8424 | 0.8466 | |
| | [90-0 days] | [0.8325 ,0.8498] | [0.8342, 0.8520] | [0.8394, 0.8493] | [0.8305, 0.8536] | [0.8394, 0.8535] | |
| PR-AUC | SaMD 40+ | 0.4958 | 0.5004 | 0.5093 | 0.4948 | 0.5071 | |
| | [90-0 days] | [0.4759, 0.5157] | [0.4829, 0.5179] | [0.4892, 0.5294] | [0.4927, 0.4969] | [0.4860, 0.5281] | |
| | SaMD 18+ | 0.4544 | 0.4609 | 0.4728 | 0.4546 | 0.4658 | |
| | [30-30 days] | [0.4321, 0.4768] | [0.4424, 0.4794] | [0.4589, 0.4867] | [0.4332, 0.4762] | [0.4441, 0.4877] | |
| | 2002-2020 | | | | | | |
| | SaMD 18+ | 0.4938 | 0.4984 | 0.5077 | 0.4928 | 0.5053 | |
| | [90-0 days] | [0.4754, 0.5122] | [0.4811, 0.5156] | [0.4866, 0.5287] | [0.4893 ,0.4962] | [0.4849, 0.5257] | |
| F1 score | SaMD 40+ | 0.5076 | 0.5036 | 0.5152 | 0.5110 | 0.5042 | |
| | [90-0 days] | [0.4960, 0.5192] | [0.4895, 0.5177] | [0.5001, 0.5303] | [0.5061, 0.5159] | [0.4925, 0.5159] | |
| | SaMD 18+ | 0.4800 | 0.4774 | 0.4914 | 0.4785 | 0.4805 | |
| | [30-30 days] | [0.4638, 0.4962] | [0.4582 ,0.4967] | [0.4817, 0.5011] | [0.4626, 0.4945] | [0.4748, 0.4862] | |
| | 2002-2020 | | | | | | |
| | SaMD 18+ | 0.5062 | 0.5020 | 0.5143 | 0.5094 | 0.5027 | |
| | [90-0 days] | [0.4957, 0.5167] | [0.4880, 0.5159] | [0.4989, 0.5295] | [0.5035, 0.5153] | [0.4903,0.5152] | |
| | | | | AF + Base | AF + Base | | |
| | | ECG only + | AF + Base | CNN (5s) + | CNN (7s) + | | |
| | | ECG/age/sex | CNN (5s) | ResNet | ResNet | Windowed | 10 Models |
| | | ensemble | ensemble | ensemble | ensemble | ensemble | (seeds, 5s) |
| # models | — | 2 | 2 | 3 | 3 | 9 | 10 |
| AUC-ROC | SaMD 40+ | 0.8306 | 0.8373 | 0.8394 | 0.8399 | 0.8360 | 0.8360 |
| | [90-0 days] | [0.8226, 0.8383] | [0.8302, 0.8441] | [0.8332, 0.8453] | [0.8336, 0.8460] | [0.8295, 0.8423] | [0.8293, 0.8426] |
| | SaMD 18+ | 0.8488 | 0.8560 | 0.8578 | 0.8589 | 0.8551 | 0.8543 |
| | [30-30 days] | [0.8390, 0.8582] | [0.8457, 0.8658] | [0.8476, 0.8674] | [0.8493, 0.8679] | [0.8455, 0.8643] | [0.8444, 0.8636] |
| | 2002-2020 | | | | | | |
| | SaMD 18+ | 0.8434 | 0.8503 | 0.8522 | 0.8527 | 0.8491 | 0.8492 |
| | [90-0 days] | [0.8360, 0.8505] | [0.8435, 0.8568] | [0.8460, 0.8581] | [0.8463, 0.8589] | [0.8427, 0.8554] | [0.8428, 0.8553] |
| PR-AUC | SaMD 40+ | 0.5050 | 0.5198 | 0.5248 | 0.5260 | 0.5142 | 0.5142 |
| | [90-0 days] | [0.4848, 0.5251] | [0.4979, 0.5416] | [0.5057, 0.5438] | [0.5079, 0.5440] | [0.4972, 0.5312] | [0.4917, 0.5366] |
| | SaMD 18+ | 0.4634 | 0.4815 | 0.4867 | 0.4886 | 0.4742 | 0.4749 |
| | [30-30 days] | [0.4407, 0.4862] | [0.4633, 0.4997] | [0.4665, 0.5068] | [0.4693, 0.5079] | [0.4570, 0.4915] | [0.4524, 0.4975] |
| | 2002-2020 | | | | | | |
| | SaMD 18+ | 0.5027 | 0.5183 | 0.5234 | 0.5245 | 0.5125 | 0.5124 |
| | [90-0 days] | [0.4831, 0.5222] | [0.4970, 0.5396] | [0.5043, 0.5424] | [0.5062, 0.5428] | [0.4960, 0.5290] | [0.4904, 0.5343] |
| F1 score | SaMD 40+ | 0.5101 | 0.5245 | 0.5217 | 0.5242 | 0.5119 | 0.5149 |
| | [90-0 days] | [0.5006, 0.5196] | [0.5172, 0.5318] | [0.5066, 0.5366] | [0.5188, 0.5295] | [0.4949 ,0.5288] | [0.5026, 0.5271] |
| | SaMD 18+ | 0.4842 | 0.4989 | 0.4974 | 0.4994 | 0.4878 | 0.4926 |
| | [30-30days] | [0.4733, 0.4951] | [0.4868, 0.5110] | [0.4849, 0.5100] | [0.4898, 0.5091] | [0.4740, 0.5017] | [0.4781, 0.5071] |
| | 2002-2020 | | | | | | |
| | SaMD 18+ | 0.5083 | 0.5236 | 0.5207 | 0.5232 | 0.5106 | 0.5134 |
| | [90-0 days] | [0.4995, 0.5171] | [0.5159, 0.5314] | [0.5055, 0.5360] | [0.5176,0 .5289] | [0.4927, 0.5285] | [0.5011, 0.5257] |

The column labeled “1 model (seed 2; 5 s)” corresponds to a base CNN architecture trained with a seed of 2, with 5 second ECG segments. The column labeled “1 model (seed 64000; 7 s)” corresponds to a base CNN architecture trained with a seed of 64000, with 7 second ECG segments. The column labeled “1 model AF arch” corresponds to an Inception-based architecture. The column labeled “1 model ResNet arch” corresponds to a ResNet architecture. The column labeled “2 models (5 s)” corresponds to an ensemble of two base CNNs, trained and tested with 5 second ECG segments. The column labeled “ECG only+ECG/age/sex/ensemble” corresponds to an ensemble of the base CNN architecture which is trained with ECG, age, and sex ensembled with the base CNN architecture trained without age and sex. The column labeled “AF+Base CNN (5 s) ensemble” corresponds to an ensemble of the base CNN architecture with 5 second ECG segments, and the Inception architecture. The column labeled “AF+Base CNN (5 s)+ResNet ensemble” corresponds to an ensemble of the base CNN architecture with 5 second ECG segments, the Inception architecture, and the ResNet architecture. The column labeled “AF+Base CNN (7 s)+ResNet ensemble” corresponds to an ensemble of the base CNN architecture with 7 second ECG segments, the Inception architecture, and the ResNet architecture. The column labeled “Windowed ensemble” corresponds to an ensemble of all of the windowed models, which were trained with the base CNN architecture. It includes 10 models, trained with the following windows: 2, 3, 4, 5, 6, 7, 8, 9, 10 seconds. The column labeled “10 models (seeds, 5 s)” corresponds to an ensemble of 10 models with the base CNN architecture, each trained with a different seed.

The rows of Table 1 correspond to different performance metrics and test groups. Table 1 includes area under the receiver operating characteristic curve (i.e., AUC-ROC) for various ensemble models. Table 1 also includes test results for precision-recall area under the curve (i.e., “PR-AUC”), and the harmonic mean of precision and recall (i.e., “F1 Score”). For each of these metrics, a higher score indicates better performance. The rows of Table 1 are further divided by test group.

As shown in Table 1, “SaMD 40+ [90-0 days]” refers to a test phenotype including subjects of age 40+ and including the most recent ECG data in a time window from 0 to 90 days before a positive or negative PH diagnosis (echo or RHC). “SaMD 18+ [90-0 days] 2002-2020” refers to a test phenotype including subjects of age 18+ and including the nearest ECG to positive diagnosis of PH in a time window from 30 before to 30 days after a positive or the nearest ECG to a negative diagnosis of PH in a time window of −infinity to 0 days, all censored to the years 2002-2020. “SaMD 18+ [90-0] days” refers to a test phenotype including subjects of age 18+ and including the most recent ECG data in a time window from 0 to 90 days before a positive or negative PH diagnosis (echo or RHC).

In some embodiments, when a subject has an RHC or echo test result that positively indicates PH at any point in their electronic health record (i.e., “EHR”), are labeled as positive. For PH-positive subjects, the date of their most recent positive RHC/echo is selected. In some embodiments, a subject is labelled as negative if the subject's EHR data does not include a RHC or echo test result that positively indicates PH. In some embodiments where the subject is negative, the most recent RHC or echo date is selected. In some embodiments, RHC data is prioritized over echo data. In one non-limiting example, when a subject's EHR data includes RHC data, the RHC data is used. If a subject's EHR data does not include RHC data but does include echo data, echo data is used.

As can be seen in Table 1, the ensemble model including a base CNN, ResNet, and Inception AI model (column “AF+Base CNN (5 s)+ResNet ensemble”) demonstrates superior performance to the ensemble including 10 base CNN models (column “10 models (seeds, 5 s)”), with an AUC-ROC for 40+ [90-0 days] of 0.8394 compared to 0.8360 for the 10 model ensemble. In general, the test results of Table 1 demonstrate that inclusion of a variety of AI model types in the ensemble improves performance of the ensemble on the task of predicting pulmonary hypertension based on ECG data. The ensembles including a variety of AI model types are also typically smaller and use fewer computing resources for training and inference than larger ensembles of a same type of AI model, such as the 10-model ensemble of base CNNs.

While Table 1 illustrates test results for the phenotypes and architectures described above, the disclosure is not so limited. In various embodiments, techniques described herein are applied to any phenotype and any AI model framework or combination thereof.

FIG. 1B is a diagram 100b illustrating segmentation of electrocardiogram data in some embodiments. As shown in FIG. 1A, ECG data segment 104d includes start sample 1,050 and end sample 4,550. Accordingly, ECG data segment 104d has a window size of 3,500 samples. While only one ECG data segment is shown in FIG. 1B, multiple ECG data windows are typically produced.

In some embodiments, ECG data windows do not overlap. In one non-limiting example, a first ECG data window includes samples 0 to 999, a second ECG data window includes samples 1,000 to 1,999, etc.

In some embodiments, ECG data windows overlap. In one non-limiting example, a first ECG data window includes samples 0 to 3,500, a second ECG data window includes samples 350 to 3,850, etc.

As discussed herein, the window size, step size, or both, are not necessarily the same for each ECG data window of segmented ECG data 104.

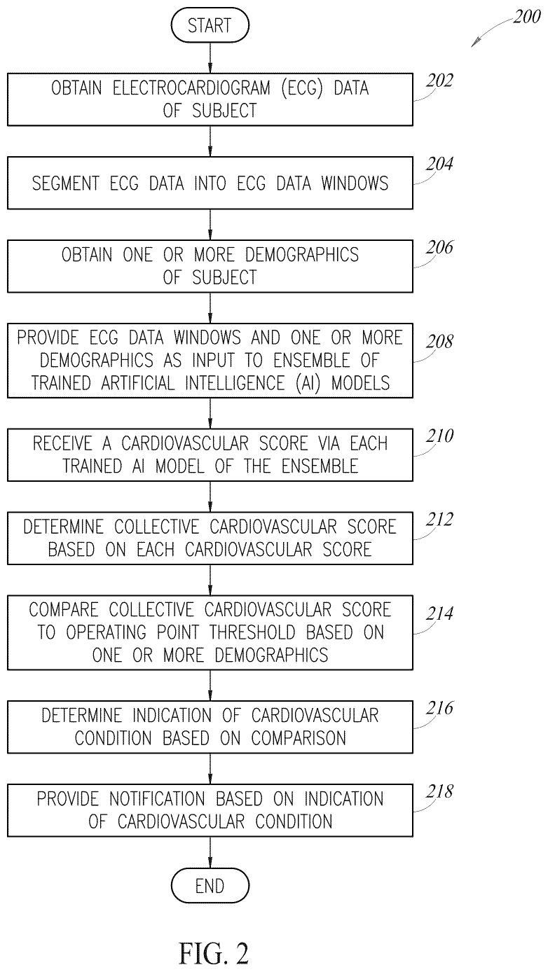

FIG. 2 is a logical flow diagram illustrating a process 200 for predicting a cardiovascular condition using an ensemble of artificial intelligence models in some embodiments. In various embodiments, process 200 is implemented using system 100a of FIG. 1A.

Process 200 begins, after a start block, at block 202, where ECG data of a subject is obtained. In some embodiments, the ECG data is obtained while the ECG data is being obtained from the subject using an ECG machine. In some embodiments, the ECG data is preexisting ECG data obtained from a database. After block 202, process 200 continues to block 204.

At block 204, the ECG data is segmented into ECG data windows (i.e., “ECG data segments”). In some embodiments, the ECG data is segmented so that each ECG data window includes a selected number of heartbeats. In some embodiments, the ECG data is segmented into a plurality of ECG data window sets according to a corresponding plurality of window sizes and step sizes. In one non-limiting example, the ECG data is segmented into a first set of ECG data windows having window size 3,500 and step size of 350, and a second set of ECG data windows having window size 2,000, and step size of 500. In various embodiments, any step size or window size are used. In some embodiments, one or more of the ECG data windows overlap. After block 204, process 200 continues to block 206.

At block 206, one or more demographics of the subject are obtained. In various embodiments, the one or more demographics include one or more demographics of the subject include age, sex, hospital setting in which the ECG data was collected, BMI, institution at which the ECG data was collected, history of conduction disorder, history of coronary re-vascularization, history of diabetes, history of myocardial infarction, ECG machine manufacturer, ECG machine model, etc., or any combination thereof. After block 206, process 200 continues to block 208.

At block 208, the ECG data windows and the one or more demographics are provided as input to a plurality of trained AI models. In various embodiments, the plurality of trained AI models includes any number of AI models such as 2-10 AI models, 20 AI models, 50 AI models, etc. In some embodiments, differently segmented ECG data is provided to two or more trained AI models. In one non-limiting example, segmented ECG data provided to a first AI model is segmented into 5 second windows and segmented ECG data provided to the second AI model is segmented into 7 second windows. In various embodiments, the one or more demographics are provided as one or more one-hot encodings, one or more normalized values, etc., or any combination thereof. In one non-limiting example, the one or more demographics include a one-hot encoded sex and a normalized age value.

In some embodiments, the unsegmented ECG data is provided as input to one or more of trained AI models instead of the segmented ECG data. For example, as depicted in FIG. 1A, ECG data 102 is provided to first AI model 110 without being segmented.

In some embodiments, the plurality of trained AI models are implemented using a virtual machine. In various embodiments, the plurality of trained AI models are implemented using any number of computing devices. In one non-limiting example, a first trained AI model is implemented using a first computing device, and a second trained AI model is implemented using a second computing device separate from the first computing device. After block 208, process 200 continues to block 210.

At block 210, a cardiovascular score is received via each trained AI model of the ensemble of trained AI models. In some embodiments, the cardiovascular scores are continuous. In one non-limiting example, the cardiovascular scores include floating point values within a selected range. In some embodiments, the cardiovascular scores are discretized. In one non-limiting example, the cardiovascular score is a discrete value selected from a set of discrete values, such as an integer between 1 and 10 or any other range. After block 210, process 200 continues to block 212.

At block 212, a collective cardiovascular score is determined based on each cardiovascular score. In some embodiments, the collective cardiovascular score is based on an average of the cardiovascular scores such as a mean, median, or mode of the cardiovascular scores. In some embodiments, combine 140 of FIG. 1A computes a weighted combination of the cardiovascular scores. In some such embodiments, the weights for calculating the weighted combination are learned, such as by freezing weights of the ensemble model and learning the weights by comparing the weighted combination of outputs produced using system 100a of FIG. 1A to a label indicating whether ECG data 102 indicates the cardiovascular condition.

In some embodiments, the collective cardiovascular score is a weighted combination of the cardiovascular scores. For example, one or more weights may be associated with each score produced by a trained AI model. In some embodiments, the weights are determined based on the number of trained AI models in the ensemble model. For example, when the ensemble model includes five trained AI models, each weight may be 0.2. In some embodiments, the weights are based on a type of each AI model in the ensemble model. For example, a first type of AI model may be associated with a weight of 0.4, and a second type of AI model may be associated with a weight of 0.6. In some embodiments, the weights are based on a performance metric of the AI model, a size of the AI model such as number of weights associated with the AI model, etc.

In some embodiments, the weights comprise one or more output layers, which compute the collective cardiovascular score based on the cardiovascular scores. In some embodiments, after each of the trained AI models is trained, weights associated with the one or more output layers are trained in a second training phase. In some such embodiments, weights of the trained AI models are frozen, and the output layers are trained based on a comparison of the determination of the cardiovascular condition. Thus, the prediction combination module may be trained to combine the cardiovascular scores into the overall cardiovascular score.

In some embodiments, the collective cardiovascular score is determined based on an ensemble voting technique, where each cardiovascular score is used to generate a vote for or against the presence of the cardiovascular condition, and the collective cardiovascular score is determined based on the votes.

In some embodiments, each cardiovascular score is compared to a threshold to determine a corresponding vote. In one non-limiting example, when a first cardiovascular score is 0.51, the first cardiovascular score is converted into a binary vote by comparing it to a selected threshold such as 0.50. When the cardiovascular score satisfies the threshold, the vote is a first binary value, such as 1. When the cardiovascular score does not satisfy the threshold, the vote is a second binary value, such as 0.

In some embodiments, the collective cardiovascular score is determined based on a majority vote. In one non-limiting example where three trained AI models are used, when a first vote is 1 (i.e., “yes”), a second vote is 1, and a third vote is 0 (i.e., “no”), the collective cardiovascular score is 1, reflecting the majority vote. In various embodiments, the collective cardiovascular score is determined based on any voting scheme.

In various embodiments, the collective cardiovascular score is a combination of the cardiovascular scores based on a number of the cardiovascular scores that indicate the presence of the cardiovascular condition. In some embodiments, a combination of the cardiovascular scores is modified according to the number of cardiovascular scores that satisfy a threshold. After block 212, process 200 continues to block 214.

At block 214, the collective cardiovascular score is compared to an operating point threshold based on the one or more demographics of the subject.

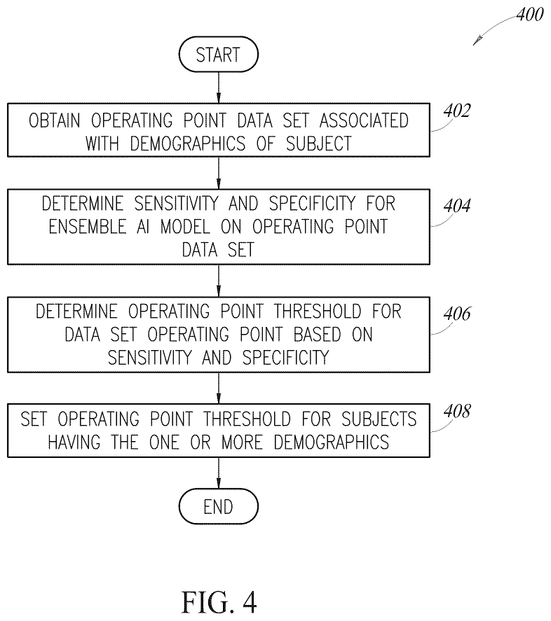

In some embodiments, the operating point threshold is determined by retrieving the operating point threshold that corresponds to the one or more demographics of the subject. In some embodiments, the operating point threshold is selected to minimize a difference between a sensitivity and specificity of the ensemble model with respect to subjects having the one or more demographics. Selection of operating point thresholds is described in detail with respect to FIG. 4. In some embodiments, the operating point threshold is determined based on one or more configurable settings. In one non-limiting example, a user such as a healthcare provider uses the one or more configurable settings to select a mode, such as a more or less sensitive mode, and the operating point threshold is determined based on the mode. After block 214, process 200 continues to block 216.

At block 216, an indication of a cardiovascular condition is determined based on the comparison of the collective cardiovascular score and the operating point threshold. In some embodiments, the indication of the cardiovascular is a binary value. For example, the indication of the cardiovascular condition indicates whether the ECG indicates pulmonary hypertension or does not indicate pulmonary hypertension.

In some embodiments, the indication of the cardiovascular condition includes a multi-class classification. For example, the ECG data may be classified as very low risk, low risk, medium risk, high risk, or very high risk with respect to the cardiovascular condition. In various embodiments, the indication of the cardiovascular condition classifies the ECG into one or more of any number of classes. For example, pulmonary hypertension may be caused by various conditions. The World Health Organization classifies cases of pulmonary hypertension into five groups by cause: (1) pulmonary arterial hypertension; (2) pulmonary hypertension due to left heart disease; (3) pulmonary hypertension due to lung disease; (4) chronic thromboembolic pulmonary hypertension; or (5) pulmonary hypertension with unknown cause. Accordingly, in some embodiments where pulmonary hypertension is the cardiovascular condition, the indication of the cardiovascular condition corresponds to one of the five groups. In some embodiments, a binary determination is made for each class based on comparing a value created for each class to an operating point. In some embodiments, each class corresponds to one or more operating points. In one non-limiting example, a collective cardiovascular score is produced for each of the classes at block 212, and an operating point is selected for comparison to each of the collective cardiovascular scores at block 214. Based on the comparisons, an indication of each class corresponding to the cardiovascular condition is produced. For example, in classifying the pulmonary hypertension into one of the five groups, the indications may be: [0, 1, 0, 0, 0], indicating that the ECG is classified into the second group, “pulmonary hypertension due to left heart disease.” After block 216, process 200 continues to block 218.

At block 218, a notification based on the indication of the cardiovascular condition is provided. In various embodiments, the notification includes a short message service (i.e., “SMS”) message, an email, a popup notification, a phone call, an audio-based notification, etc. In various embodiments, the notification is provided to the subject, a healthcare provider providing care to the subject, etc.

As discussed herein, process 200 may be performed in real-time or near real-time when an ECG measurement is being taken for the subject. This may enable care providers to more expeditiously or efficiently address the cardiovascular condition because the subject is already at the care facility, improving outcomes. A notification based on the indication of the cardiovascular condition may be used to recommend performance of a diagnostic test such as an echocardiogram (i.e., an “echo”), a right heart catheterization, etc. to confirm whether the subject has the indicated cardiovascular condition. In some embodiments, the notification includes automatically scheduling or recommending that the subject be scheduled a diagnostic test, alerting a care provider that the subject may have the cardiovascular condition, etc. After block 218, process 200 ends at an end block.

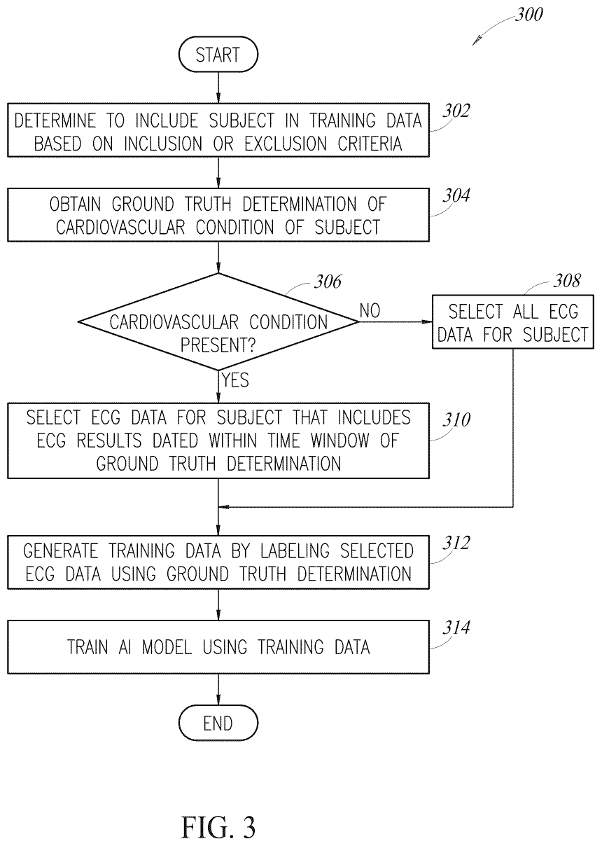

FIG. 3 is a logical flow diagram illustrating a process for selecting training data for training an artificial intelligence model in some embodiments. In various embodiments, process 300 is used to select training data for training one or more of first AI model 110, second AI model 120, or third AI model 130 of FIG. 1A.

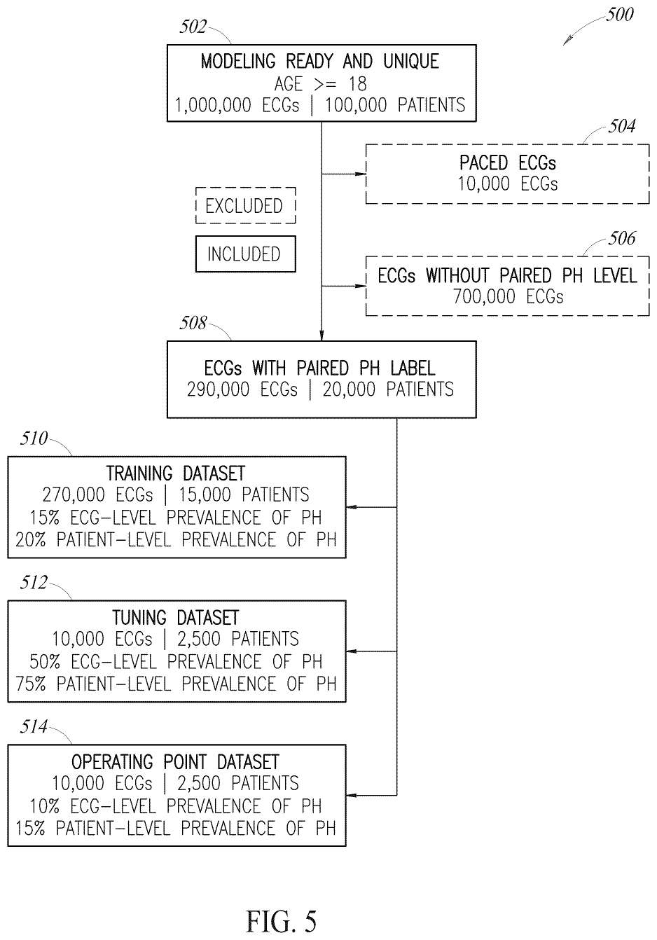

Process 300 begins, after a start block, at block 302 where a determination is made to include or exclude data associated with a subject in training data. Inclusion and exclusion criteria are now discussed with respect to FIG. 5.

FIG. 5 is block diagram 500 illustrating dataset creation for training an artificial intelligence model in some embodiments. In FIG. 5, solid blocks are included in a dataset, while dashed blocks are not included in the dataset.

In some embodiments, modeling ready and unique ECGs 502 are ECGs taken from subjects 18 years or older and are not duplicative of any other ECGs. In some embodiments, modeling ready and unique ECGs 502 include multiple ECGs for one subject. For example, one subject may have multiple samples of ECG data available corresponding to several ECG tests. In the example shown in FIG. 4, 1,000,000 ECGs are included from 200,000 subjects.

Paced ECGs 504 include ECGs taken from subjects having active pacemakers. In some embodiments, paced ECGs 504 are excluded from the dataset.

ECGs without paired pulmonary hypertension (PH) level 506 include ECGs that are not paired with a corresponding PH level. While the example shown in FIG. 5 relates to pulmonary hypertension, the disclosure is not so limited. As discussed herein, in various embodiments data associated with any cardiovascular condition is used to label the ECGs. In some embodiments, any condition that can be indicated by ECG data is used to label the ECGs. Because functioning of systems of a subject's body are often closely related to cardiovascular function, ECG data is often correlated with conditions in various bodily systems. For example, pulmonary conditions such as pulmonary hypertension may be indicated by ECG data. Accordingly, when another condition is to be predicted, ECG data without a paired label regarding that condition may be excluded. For example, when an AI model is to predict coronary artery disease, ECGs without paired labels regarding coronary artery disease may be excluded.

ECGs with paired PH label 508 include ECGs that are paired with a PH label. In some embodiments, an ECG is paired with a PH label when the ECG is within a selected time window of a positive or negative PH diagnosis.

ECGs with paired PH label 508 is separated into training dataset 510, tuning dataset 512, and operating point dataset 514.

Training dataset 510 is configured to be usable to train an AI model to predict a label for a relevant condition. As shown in FIG. 5, training dataset 510 includes 270,000 ECGs from 15,000 subjects, with a 15% ECG-level prevalence of PH and a 20% subject-level prevalence of PH.

Tuning dataset 512 is configured to be usable to determine when to cease training an AI model. As shown in FIG. 5, tuning dataset 512 includes 10,000 ECGs from 2,500 subjects, with a 50% ECG-level prevalence of PH and a 75% subject-level prevalence of PH.

Operating point dataset 514 is configured to be usable to determine operating points for the relevant condition. As shown in FIG. 5, operating point dataset 514 includes 10,000 ECGs from 2,500 subjects, with a 10% ECG-level prevalence of PH and a 15% subject-level prevalence of PH.

Returning to FIG. 3, after the determination is made to include or exclude data associated with a subject, process 300 continues to block 304, where a ground truth determination of a cardiovascular condition of the subject is obtained. In various embodiments, the ground truth determination is from clinical notes, one or more test results, etc. In one non-limiting example, a ground truth determination for the presence of pulmonary hypertension is based on an echocardiogram test result.

In various embodiments, the ground truth determination is based on comparing one or more test results to one or more selected test result thresholds. In one non-limiting example wherein the cardiovascular condition is pulmonary hypertension, a determination that ECG data positively indicates pulmonary hypertension is based on a right-heart catheterization test result above a first selected threshold, an echo result above a second selected threshold, or both.

In one non-limiting example where the cardiovascular condition is pulmonary hypertension (PH), PH ground truth labels are based on echocardiogram (‘echo’) reports, right heart catheterization (RHC) reports, clinical notes or any combination thereof. Subjects with available RHC data are labeled as positive if they have an mPAP>20 mmHg. Subjects without available RHC data with at least one echo are labeled as positive if any of their echos include a tricuspid regurgitant velocity (i.e., “TRV”) greater than a threshold value such as 3.4 m/sec. In some embodiments, subjects without RHC or echo test results are labeled as positive if there is evidence of PH in their clinical notes. In some embodiments, subjects are labeled as negative if each available RHC has mean pulmonary arterial pressure (i.e., “mPAPs”) less than a threshold value such as 20 mmHgs (where applicable), or if they never had RHC and each echo TRV measurements were below a threshold value such as 2.8 m/s. In some embodiments, subjects with TRV measurements >2.8 m/sec and ≤3.4 m/sec are considered indeterminate and excluded. In some embodiments, clinical notes are not used to create negative labels.

In various embodiments, the ground truth determination is created based on a natural language processing (i.e., “NLP”) label created using an NLP model. In some embodiments, the NLP model takes text such as diagnosis codes, problem lists, procedures, medications, imaging results, or any combination thereof, as input. In some embodiments, the NLP model is based on one or more regular expressions, artificial intelligence models, classifiers, logical rules, or any combination thereof. The NLP model produces as output a “weak label” indicative of whether the text indicates that a subject is likely to have the cardiovascular condition. NLP labels may be used, for example, to provide a ground truth label for ECG data for which a corresponding test-based ground truth label is unavailable. In some embodiments, NLP labels are used to augment training data so ECG data having test-based ground truth can be held out for use in a testing or validation data set. Embodiments of the NLP model and NLP labels are described in further detail in U.S. application Ser. No. 18/393,312, filed Dec. 21, 2023 and entitled “SYSTEMS, METHODS, AND ARTICLES FOR ENHANCING THE TRAINING OF NATURAL LANGUAGE PROCESSING MODELS IN A BIOMEDICAL CONTEXT,” which is hereby incorporated by reference in its entirety.

In some embodiments, the ground truth determinations include a combination of ground truth determinations based on test results and ground truth determinations based on an NLP model. In some embodiments, the ground truth determinations based on test results are used to label ECG data when they are available, and the NLP labels are used to label the ECG data when the test results are unavailable.

In some embodiments, the ground truth labels are determined using a label according to a label preference hierarchy. As described herein, labels may be created using various processes and various data sources. Some processes or data sources may be preferred over others because they are more reliable, more efficient to obtain, etc. In one non-limiting example where the cardiovascular condition is pulmonary hypertension, the label preference hierarchy is, from most preferred to least preferred: (1) labels produced based on right heart catheterization test results; (2) labels produced based on echocardiogram results; and (3) labels produced based on an NLP model. Often, labels based on tests such as echocardiograms or right heart catheterization are considered more definitive than labels created using other methods and are therefore preferably used when available. But in various embodiments, any label preference hierarchy is used.

At decision block 306, a determination is made whether the ground truth determination indicates that the cardiovascular condition is present. If yes, process 300 proceeds to block 310. If no, process 300 proceeds to block 308.

At block 308, when the cardiovascular condition is not present, all ECG data for the subject is selected. In one non-limiting embodiment, when a subject is determined not to have pulmonary hypertension based on the ground truth determination, all ECG data available for the subject is selected. After block 308, process 300 continues to block 312.

At block 310, when the cardiovascular condition is present, ECG data for the subject that includes ECG results dated within a time window of the ground truth determination is selected. In some embodiments wherein a subject is determined to have a cardiovascular condition, some ECG samples may exist for the subject from a time period before onset of the cardiovascular condition. Because these ECG results may not be predictive of the cardiovascular condition, ECG results within a time period of the ground truth determination are selected. In one non-limiting example, ECG results within 90 days of a positive diagnosis of pulmonary hypertension are included, and ECG results outside of 90 days of the diagnosis are excluded. In various embodiments, any time window is used. After block 310, process 300 proceeds to block 312.

At block 312, training data is generated by labeling selected ECG data using the ground truth determination. In one non-limiting example, when the ground truth determination indicates that a subject does not have pulmonary hypertension, ECG data of the subject is labeled “0”; when the ground truth determination indicates that a subject does have pulmonary hypertension, ECG data of the subject is labeled “1”. After block 312, process 300 proceeds to block 314.

At block 314, an AI model is trained using the training data. In some embodiments, training the AI model includes providing ECG data and one or more demographics of the subject as input to the AI model. The output of the AI model is compared to a ground truth label for the cardiovascular condition. Weights of the AI model are modified based on the comparison, such as by backpropagation. In one non-limiting example, ECG data and indications of age and sex are provided to the AI model as input. Output of the AI model is compared to a ground truth label of the cardiovascular condition, such as pulmonary hypertension. After block 314, process 300 ends at an end block.

While process 300 is described in terms of selecting training data for training a single AI model, the disclosure is not so limited. In some embodiments, training data for training multiple AI models is selected. In one non-limiting example, training data for each of AI models 110, 120, and 130 of FIG. 1A is selected. In some embodiments, process 300 is used to obtain multiple sets of training data to train corresponding AI models.

FIG. 4 is a logical flow diagram illustrating a process 400 used to determine an operating point threshold for subjects having one or more demographics according to some embodiments. In classification problems, sensitivity refers to the true positive rate of a classifier, while specificity refers to the true negative rate of the classifier. For example, a classifier that classifies every subject as having a cardiovascular condition would have a sensitivity of 1 because every subject that has the cardiovascular condition is classified as having cardiovascular condition. But such a classifier would also typically have poor specificity because all subjects not having the cardiovascular condition are also classified as having the cardiovascular condition. Typically, a balance between sensitivity and specificity is preferable, such that the ensemble model is not substantially overinclusive or underinclusive in determining an indication of a cardiovascular condition.

Sensitivity and specificity of the ensemble model may be adjusted by changing the operating point threshold. The operating point threshold is used to transform the collective cardiovascular score calculated by the ensemble model into an indication of whether the subject has a cardiovascular condition. For example, a lower operating point threshold may lead to higher sensitivity and lower specificity because more subjects are classified as having the cardiovascular condition, while a higher operating point threshold may lead to lower sensitivity and higher specificity. In various embodiments, block 214 of FIG. 2 employs embodiments of process 400 to obtain the operating point threshold for the subject based on one or more demographics of the subject.

Process 400 begins, after a start block, at block 402, where an operating point dataset associated with demographics of the subject is obtained. In various embodiments, the operating point dataset is obtained to include ECG data and corresponding labels from subjects having one or more demographics. For example, when the operating point thresholds to be calculated are based on the binarized sex of subjects, an operating point dataset corresponding to subjects with female sex is obtained, and an operating point dataset corresponding to subjects with male sex is obtained. In some embodiments, the operating point dataset includes a configurable proportion of each class of a binarized or otherwise quantized characteristic. For example, an operating point dataset associated with binarized sex may be include 50% male datapoints and 50% female datapoints. In some embodiments, the operating point dataset includes a selected prevalence of ECGs from subjects in which a cardiovascular condition is indicated. The prevalence may be based on a prevalence of the cardiovascular condition in the training condition. In one non-limiting example, when the training dataset includes a 10% prevalence of ECGs from middle-aged males having pulmonary hypertension (i.e., “PH”), the operating point dataset is constructed to include a 10% prevalence of ECGs from middle-aged males having PH.

In some embodiments, binarization of subject demographics into two classes is determined based on a threshold value. In one non-limiting example, age is binarized into younger subjects for subjects younger than age 65 and older subjects for subjects 65 and older. In another example, body mass index (i.e., “BMI”) is binarized into a group of low BMI and a group of high BMI, where BMIs above a 90th percentile of BMIs are in the group of high BMIs, and where BMIs below the 90th percentile are in the group of low BMIs.

In some embodiments, one or more demographics are quantized into more than two discrete groups. In one non-limiting example, BMI is quantized into four groups such as BMIs less than 18.5, BMIs between 18.5 and 25, BMIs between 25 and 30, and BMIs greater than 30. In another example, institution is quantized into several groups corresponding to each of the institutions that contributed ECG data to the dataset.

In various embodiments, an operating point dataset is obtained based on any combination of the one or more quantized demographics. In one non-limiting example, age is quantized into three discrete groups such as “younger,” “middle-aged,” and “older,” and smoking status is quantized into two discrete groups such as “smokes” and “does not smoke.” Accordingly, an operating point threshold dataset may be obtained such that it includes subjects with the combination of the one or more quantized demographics such as “younger” and “does not smoke.” After block 402, process 400 continues to block 404.

At block 404, a sensitivity and specificity for an ensemble AI model on the operating point dataset is determined. In some embodiments, the sensitivity and specificity for the ensemble model on the operating point dataset is determined by providing ECG data of the operating point dataset as input to the ensemble model, and comparing the output of the ensemble model to corresponding ground truth labels. Referring to FIG. 1A by way of example, ECG data 102 is provided as input to the ensemble model, which eventually produces cardiovascular prediction 146. Cardiovascular prediction 146 is compared to a label that corresponds to the ECG data of the operating point dataset to determine whether the ensemble model correctly predicted the cardiovascular condition. By performing inference on a plurality of ECG data samples of the operating point dataset and comparing the results to the corresponding labels, a sensitivity and specificity score of the ensemble model on the operating point dataset are produced.

In some embodiments, the sensitivity and specificity comprise a receiver operating characteristic (ROC) graph. In some embodiments, the sensitivity and specificity include multiple pairs of corresponding sensitivity and specificity values. After block 404, process 400 continues to block 406.

At block 406, an operating point threshold is determined for the operating point dataset based on the sensitivity and specificity. In some embodiments, the operating point threshold is determined to optimize a Youden's index computed using the sensitivity and specificity. In some embodiments, the operating point threshold is determined to optimize an absolute difference between the sensitivity and the specificity. In some embodiments, the operating point threshold is determined to optimize an F1 score calculated using the sensitivity and specificity. In some embodiments, the operating point threshold is determined to optimize an F2 score calculated using the sensitivity and specificity. In various embodiments, the operating threshold is determined to optimize any metric based on the sensitivity and the specificity. In various embodiments, the operating threshold is determined to optimize any other metric, such as positive predictive value (i.e. “PPV”), etc. In various embodiments the operating point threshold is selected to achieve a selected target such as a sensitivity of 0.5. After block 406, process 400 continues to block 408.

At block 408, an operating point threshold is set for subjects having the one or more demographics. Accordingly, when a collective cardiovascular score is determined using ECG data associated with a subject having the one or more demographics, the operating point threshold is used to determine whether the overall cardiovascular score indicates that the subject has the cardiovascular condition.

While process 400 is described in terms of determining a single operating point threshold, the disclosure is not so limited. In various embodiments, process 400 is used to determine any number of operating point thresholds. In one non-limiting example, an operating point is established for each of several demographic groups such as sex and age. In one non-limiting example, an operating point is younger females (ages 18 to 39 years), middle-aged females (ages 40 to 65 years), older females (65+ years), younger males (ages 18 to 39 years), middle-aged males (ages 40 to 65 years), older males (65+ years). In some embodiments, an operating point dataset is obtained for each of the demographic groups, sensitivity and specificity of the ensemble model are determined using each operating point dataset, and an operating point threshold for each demographic group is determined based on the sensitivity and specificity.

FIG. 6 is a block diagram illustrating a first artificial intelligence model architecture 600 in some embodiments. As shown in FIG. 6, the first artificial intelligence model architecture is based on a base convolutional neural network (CNN) architecture. The base CNN architecture includes one or more convolutional blocks configured to extract features from time-series data such as segmented ECG data 104. The base CNN architecture typically includes fewer parameters than other architectures, such as Inception or ResNet. Accordingly, the base CNN architecture may be less likely to overfit than other architectures.

First AI model architecture 600 includes convolutional blocks 602, subject demographic block 604, and fully connected dense layers 606.

Segmented ECG data 104 is provided to convolutional blocks 602, and subject demographics 106 is provided to subject demographics block 604. Outputs of convolutional blocks 602 and subject demographic block 604 are provided as input to fully connected dense layers 606, which produce output 608, such as second cardiovascular score 126.

As shown in FIG. 6, convolutional blocks 602 include 6 convolutional blocks. In various embodiments, convolutional blocks 602 include any number of convolutional blocks. In some embodiments, convolutional blocks are connected in series.

In some embodiments, convolutional blocks 602 are configured to receive 7-second segments of ECG data as input. In various embodiments the sub-blocks included in convolutional blocks 602 vary, the configuration of the sub-blocks vary, or both. For example, convolutional blocks 602 are depicted as including subblocks such as a 1D convolutional layer, a batch normalization layer, a ReLU activation function, etc. In various embodiments, convolutional blocks 602 include any combination of convolutional layers, batch normalization layers, activation functions, max pooling layers, etc.

In some embodiments, subject demographics 106 include an indication of one or more of age, sex, height, weight, location of residency, etc. In some embodiments, the output of subject demographic block 604 and convolutional blocks is concatenated before being provided as input to fully connected dense layers 606. Fully connected dense layers 606 produce output 608, such as second cardiovascular score 126 of FIG. 1A.

While subject demographic block 604 is depicted as including a fully connected dense layer and a ReLU activation function, in various embodiments any number of dense fully connected layers, normalization layers, or activation functions, or any combination thereof, are used.

FIG. 7 is a block diagram illustrating a second artificial intelligence model architecture in some embodiments. As shown in FIG. 7, second artificial intelligence architecture 700 is based on an inception architecture. Inception is a type of CNN architecture typically characterized by inception blocks, which include filters of various sizes. Because Inception blocks include filters of various sizes, inception-based AI models are useful for extracting hierarchical or multi-scale features from input data.

Second AI architecture 700 includes convolutional block 702, inception blocks 704, GAP layer 706, subject demographics block 604, concatenate 708, and fully connected dense layers 710. Convolutional block 702 includes a 1D convolutional layer, ReLU activation function, and a batch norm layer, in series. As shown in FIG. 7, inception blocks 704 includes four inception blocks in series, where each inception block comprises three 1D convolutional blocks concatenated across the channel axis with decreasing filter window sizes. Inception blocks 704 are connected to another single convolutional block and a global averaging pool (i.e., “GAP”) layer 706. Subject demographics 106 are input into subject demographics block 604, which in some embodiments includes one or more hidden layers that correspond to each subject demographic. The output from GAP layer 706 and the output of subject demographics block 604 are concatenated at concatenate 708. The output of concatenate 708 is passed to fully connected dense layers 710, which as shown in FIG. 7 include 4 dense layers of 64 units, 32 units, 8 units, and 1 unit, with a sigmoid function as the final layer. In some embodiments, layers in second AI architecture 700 enforce kernel constraints and have no bias terms. Fully connected dense layers 710 produces output 712, such as first cardiovascular score 116 of FIG. 1A.

FIG. 8 is a block diagram illustrating a third artificial intelligence (AI) model architecture 800 in some embodiments. As shown in FIG. 8, the third AI model architecture 800 is based on a ResNet architecture. ResNet is a type of CNN architecture. ResNet-based architectures are useful for reducing the effects of vanishing gradients in deep AI models. While increasing layers of an AI model often improves performance of the AI model on complex tasks, as the number of layers (i.e., the “depth”) of the AI model increases, exploding or vanishing gradients in the AI model tend to increase. Exploding or vanishing gradients are detrimental to the performance of the AI model. ResNet addresses this problem by introducing skip connections. Skip connections enable an activation of a first layer of the AI model to be provided to a second layer of the AI model. By skipping one or more layers of the AI model, exploding or vanishing gradients are reduced. ResNet therefore enables third AI model architecture 800 to potentially have greater depth than AI models based on other architectures such as a base CNN architecture. The increased depth enabled by ResNet architectures may enable improved performance on segmentation, feature extraction, or object detection tasks.

Third AI model architecture 800 includes convolutional blocks 802, subject demographic block 604, and fully connected dense layers 806. In some embodiments, fully connected dense layers 806, subject demographics block 604, or both, are similar to fully connected dense layers 606 or subject demographic block 604 described with respect to FIG. 6.

In some embodiments, one or more convolutional blocks in convolutional blocks 802 include with a depth of 12 convolutional layers. While convolutional blocks 802 is depicted as including 6 convolutional blocks in FIG. 8, in various embodiments convolutional blocks 802 includes any number of convolutional blocks. In some embodiment, a first convolutional block employs 8 filters, which are doubled every 2 layers, resulting in a progressive increase in the number of filters as the network depth increases. In some embodiments, each convolutional layer uses a kernel size of 7 and is followed by batch normalization and ReLU activation to ensure stable and efficient training. In some embodiments, max pooling with a pool size such as 3 is applied every 2 layers to reduce the spatial dimensions of the feature maps. In some embodiments, skip connections are introduced every 2 layers to facilitate residual learning and mitigate the vanishing or exploding gradients. After the convolutional feature extraction, the output is flattened and concatenated with additional input features, such as age and sex, and processed through a dense layer with 32 units and ReLU activation. The concatenated features are then passed through fully connected dense layers 806. In some embodiments, fully connected dense layers 806 includes two hidden layers with 128 and 64 units, respectively, each followed by dropout. A third output layer consists of a single neuron with a sigmoid activation function, suitable for binary classification tasks. Fully connected dense layers 806 produce output 808, such as third cardiovascular score 136 of FIG. 1A.

In some embodiments, third AI model architecture 800 takes segments of ECG data as input for classification. In some embodiments a sliding window approach is used to make predictions across the ECG data. This involves providing windows of the ECG data to third AI model architecture 800. In one non-limiting example where the ECG data has a full signal duration of 10 seconds, the ECG data is segmented into 7-second windows with a step size of 700 milliseconds. This allows the model to generate a prediction for each different segment of the signal. In some embodiments, the predictions for each segment are then averaged to provide one final ResNet CNN model prediction. This method may improve the robustness and accuracy of the classification by leveraging temporal information across the full duration of the ECG data.

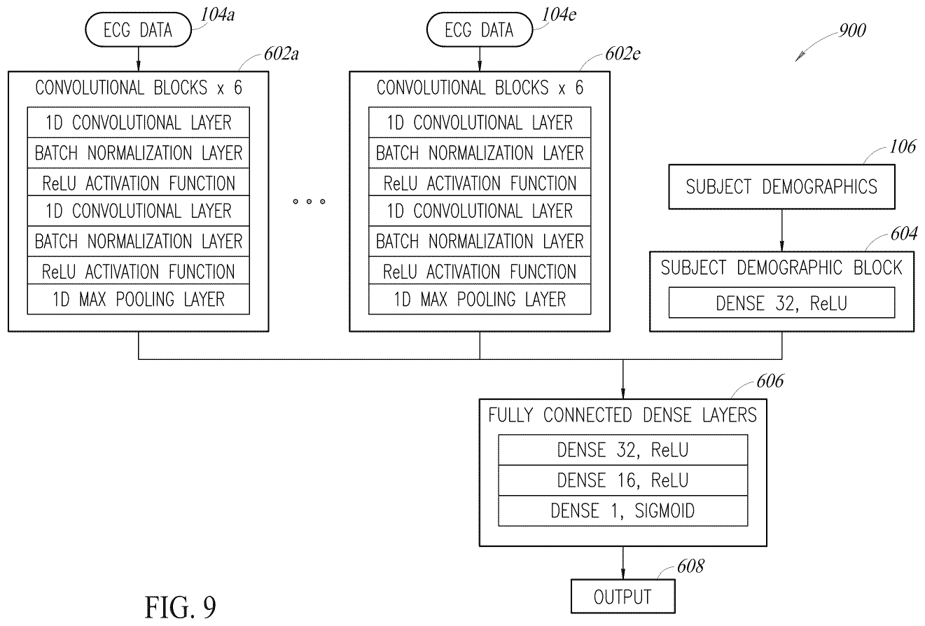

FIG. 9 is a block diagram illustrating a fourth artificial intelligence model architecture in some embodiments. Fourth AI architecture 900 includes a set of convolutional blocks for each segment of ECG data in segmented ECG data 104. As shown in FIG. 1A, segmented ECG data 104 includes five ECG data segments 104a, 104b, 104c, 104d, and 104e. Accordingly, fourth AI architecture 900 includes five sets of convolutional blocks 602a to 602e. In some embodiments, fourth AI architecture 900 is configured to enable segmented ECG data 104 to be processed in parallel. In some embodiments, one or more of convolutional blocks 602a to 602e include an inception block, a residual block, or any other artificial intelligence architecture or component thereof. Convolutional blocks 602a to 602e are not necessarily the same. In various embodiments, convolutional blocks

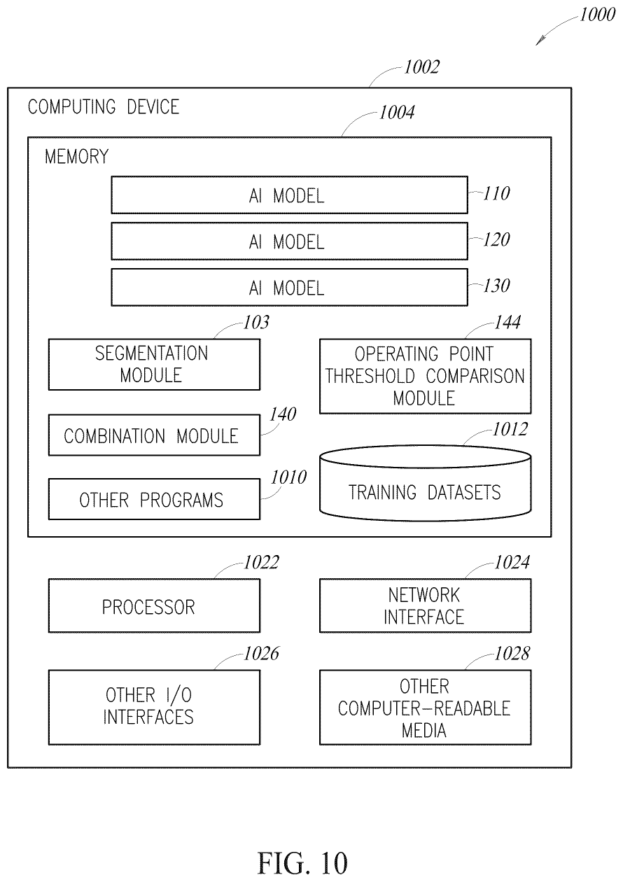

FIG. 10 shows a system diagram that describes one implementation of computing systems for implementing embodiments described herein. System 1000 includes computing device 1002.

As described herein, computing device 1002 is configured to perform functionality described herein for detecting a cardiovascular condition from electrocardiogram data using an ensemble of artificial intelligence models. One or more special purpose computing systems may be used to implement computing device 1002. Accordingly, various embodiments described herein may be implemented in software, hardware, firmware, or in some combination thereof. Computing device 1002 includes memory 1004, one or more processors 1022, network interface 1024, other input/output (I/O) interfaces 1026, and other computer-readable media 1028. In some embodiments, computing device 1002 may be implemented by cloud computing resources.

Processor 1022 includes one or more processors, processing units, programmable logic, circuitry, or other computing components that are configured to perform embodiments described herein or to execute computer instructions to perform embodiments described herein. In some embodiments, processor 1022 may include a single processor that operates individually to perform actions. In other embodiments, processor 1022 may include a plurality of processors that operate to collectively perform actions, such that one or more processors may operate to perform some, but not all, of such actions.

Memory 1004 may include one or more various types of non-volatile or volatile storage technologies. Examples of memory 1004 include, but are not limited to, flash memory, hard disk drives, optical drives, solid-state drives, various types of random-access memory (“RAM”), various types of read-only memory (“ROM”), other computer-readable storage media (also referred to as processor-readable storage media), or other memory technologies, or any combination thereof. Memory 1004 may be utilized to store information, including computer-readable instructions that are utilized by processor 1022 to perform actions, including at least some embodiments described herein.

In some embodiments, memory 1004 may have stored thereon first AI model 110, second AI model 120, third AI model 130, segmentation module 103, combination module 140, operating point threshold comparison module 144, other programs 1010, training datasets 1012, or any combination thereof.

Other programs 1010 may include operating systems, user applications, or other computer programs.

Network interface 1024 is configured to communicate with other computing devices via a communication network. Network interfaces 1024 include transmitters and receivers (not illustrated) to send and receive data.

Other I/O interfaces 1026 may include interfaces for various other input or output devices, such as audio interfaces, other video interfaces, USB interfaces, physical buttons, keyboards, haptic interfaces, tactile interfaces, etc. Other computer-readable media 1028 may include other types of stationary or removable computer-readable media, such as removable flash drives, external hard drives, etc.

The following is a summarization of the claims as filed.

In various embodiments, a method includes: receiving electrocardiogram (ECG) data of a subject, wherein the ECG data includes a time series of voltage measurements; segmenting the received ECG data into a plurality of ECG data windows; obtaining one or more demographics of the subject; providing the plurality of ECG data windows and the one or more demographics as input to: a first trained artificial intelligence (AI) model having a first framework; and a second trained AI model having a second framework different from the first framework; receiving, via the first trained AI model, a first cardiovascular score; receiving, via the second trained AI model, a second cardiovascular score; determining a collective cardiovascular score based on the first cardiovascular score and the second cardiovascular score; identifying, based on the one or more demographics, an operating point threshold from a plurality of operating point thresholds; comparing the collective cardiovascular score to the operating point threshold; determining an indication of a cardiovascular condition based on the comparison; and providing a notification based on the indication of the cardiovascular condition.

In some embodiments, determining the first cardiovascular score includes: generating, using the first trained AI model, a plurality of cardiovascular window scores, each corresponding to a different one of the plurality of ECG data windows; and determining the first cardiovascular score based on a combination of the plurality of cardiovascular window scores.

In some embodiments, the collective cardiovascular score includes a score of pulmonary hypertension, and the cardiovascular condition is pulmonary hypertension.

In some embodiments, segmenting the received ECG data is based on one or more of a step size or a window size.

In some embodiments, determining the collective cardiovascular score includes: computing an average of the first cardiovascular score and the second cardiovascular score.

In some embodiments, determining the collective cardiovascular score includes: providing the first cardiovascular score and the second cardiovascular score as input to a classifier; and determining the collective cardiovascular score based on output of the classifier.

In some embodiments, determining the indication of the cardiovascular condition includes: determining a binary prediction of whether the subject has the cardiovascular condition based on the comparison.

In some embodiments, the method further includes: providing the ECG data as input to a third trained AI model; determining, using the third trained AI model, a third cardiovascular score; and determining the collective cardiovascular score based on the third cardiovascular score.

In some embodiments, the first framework includes a ResNet architecture or an Inception architecture.

In some embodiments, the one or more demographics include a representation of sex of the subject and a representation of age of the subject.

In some embodiments, identifying the operating point threshold includes: identifying the operating point threshold from at least four possible operating points based on the one or more demographics.

In some embodiments, identifying the operating point threshold includes: identifying the operating point threshold that minimizes a difference between a sensitivity and a specificity of indications of the cardiovascular condition determined using the first trained AI model and the second trained AI model for subjects having the one or more demographics.

In some embodiments, providing the one or more demographics as input to the first trained AI model includes: providing the one or more demographics to a subject demographic block of the first trained AI model.

In some embodiments, determining the first cardiovascular score using the first trained AI model includes: providing the plurality of ECG data windows as input to a convolutional block of the first trained AI model; providing the one or more demographics as input to a subject demographic block of the first trained AI model; and determining the first cardiovascular score based on output of the convolutional block and output of the subject demographic block.

In some embodiments, determining the first cardiovascular score using the first trained AI model includes: providing the plurality of ECG data windows as input to a convolutional block of the first trained AI model; providing the one or more demographics as input to a subject demographic block of the first trained AI model; providing output of the convolutional block and output of the subject demographic block as input to one or more fully connected layers; and determining the first cardiovascular score based on output of the one or more fully connected layers.

In some embodiments, providing the notification based on the indication of the cardiovascular condition includes: providing the notification recommending that the subject be scheduled for a diagnostic test to confirm the indication of the cardiovascular condition.

In various embodiments, a system includes: one or more processors; and one or more non-transitory computer-readable media storing contents executable by the one or more processors to cause the system to: receive electrocardiogram (ECG) data of a subject, wherein the ECG data includes a time series of voltage measurements; determine a plurality of ECG data windows based on ECG data; obtain one or more demographics of the subject; provide the plurality of ECG data windows and the one or more subject demographics as input to an ensemble of trained AI models, wherein the ensemble of trained AI models includes AI models having at least two different AI model frameworks; determine a plurality of cardiovascular scores using the ensemble of trained AI models; determine a collective cardiovascular score based on the plurality of cardiovascular scores; obtain an operating point threshold based on the one or more demographics; compare the collective cardiovascular score to the operating point threshold; and determine an indication of a cardiovascular condition based on the comparison.

In some embodiments, the one or more processors provide the subject demographics as input to the plurality of trained AI models by being further configured to: provide the subject demographics to a subject demographic block of each trained AI model of the ensemble of trained AI models.

In some embodiments, one or more non-transitory computer-readable storage media collectively store contents executable by one or more processors to perform actions, the actions including: obtaining a plurality of ECG data windows based on ECG data of a subject; obtaining one or more demographics of the subject; providing the plurality of ECG data windows and the one or more demographics as input to a first trained artificial intelligence (AI) model having a first framework and to a second trained AI model having a second framework different from the first framework; determining, using the first trained AI model, a first pulmonary score; determining, using the second trained AI model, a second pulmonary score; determining an overall pulmonary score based on the first pulmonary score and the second pulmonary score; obtaining an operating point threshold based on the one or more demographics; comparing the overall pulmonary score to the operating point threshold; and determining an indication of a pulmonary condition based on the comparison.

In some embodiments, the one or more non-transitory computer-readable storage media are executable to obtain the plurality of ECG data windows based on the ECG data by: segmenting the ECG data into the plurality ECG data windows, wherein each ECG data window overlaps with at least one other ECG data window.

U.S. application Ser. No. 18/821,307, filed Aug. 30, 2024 and entitled “DETECTING LOW EJECTION FRACTION FROM ELECTROCARDIOGRAM DATA USING ARTIFICIAL INTELLIGENCE,” is hereby incorporated by reference in its entirety.

In cases where the present application conflicts with a document incorporated by reference, the present application controls.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
receiving electrocardiogram (ECG) data of a subject, wherein the ECG data includes a time series of voltage measurements;
segmenting the received ECG data into a plurality of ECG data windows;
obtaining one or more demographics of the subject;
providing the plurality of ECG data windows and the one or more demographics as input to:
a first trained artificial intelligence (AI) model having a first framework; and
a second trained AI model having a second framework different from the first framework;
receiving, via the first trained AI model, a first cardiovascular score;
receiving, via the second trained AI model, a second cardiovascular score;
determining a collective cardiovascular score based on the first cardiovascular score and the second cardiovascular score;
identifying, based on the one or more demographics, an operating point threshold from a plurality of operating point thresholds;
comparing the collective cardiovascular score to the operating point threshold;
determining an indication of a cardiovascular condition based on the comparison; and
providing a notification based on the indication of the cardiovascular condition.

2. The method of claim 1, wherein determining the first cardiovascular score includes:
generating, using the first trained AI model, a plurality of cardiovascular window scores, each corresponding to a different one of the plurality of ECG data windows; and
determining the first cardiovascular score based on a combination of the plurality of cardiovascular window scores.

3. The method of claim 1, wherein the collective cardiovascular score includes a score of pulmonary hypertension, and the cardiovascular condition is pulmonary hypertension.

4. The method of claim 1, wherein segmenting the received ECG data is based on one or more of a step size or a window size.

5. The method of claim 1, wherein determining the collective cardiovascular score includes:
computing an average of the first cardiovascular score and the second cardiovascular score.

6. The method of claim 1, wherein determining the collective cardiovascular score includes:
providing the first cardiovascular score and the second cardiovascular score as input to a classifier; and
determining the collective cardiovascular score based on output of the classifier.

7. The method of claim 1, wherein determining the indication of the cardiovascular condition includes:
determining a binary prediction of whether the subject has the cardiovascular condition based on the comparison.

8. The method of claim 1, further comprising:
providing the ECG data as input to a third trained AI model;
determining, using the third trained AI model, a third cardiovascular score; and
determining the collective cardiovascular score based on the third cardiovascular score.

9. The method of claim 1, wherein the first framework includes a ResNet architecture or an Inception architecture.

10. The method of claim 1, wherein the one or more demographics include a representation of sex of the subject and a representation of age of the subject.

11. The method of claim 1, wherein identifying the operating point threshold includes:
identifying the operating point threshold from at least four possible operating points based on the one or more demographics.

12. The method of claim 1, wherein identifying the operating point threshold includes:
identifying the operating point threshold that minimizes a difference between a sensitivity and a specificity of indications of the cardiovascular condition determined using the first trained AI model and the second trained AI model for subjects having the one or more demographics.

13. The method of claim 1, wherein providing the one or more demographics as input to the first trained AI model includes:
providing the one or more demographics to a subject demographic block of the first trained AI model.

14. The method of claim 1, wherein determining the first cardiovascular score using the first trained AI model includes:
providing the plurality of ECG data windows as input to a convolutional block of the first trained AI model;
providing the one or more demographics as input to a subject demographic block of the first trained AI model; and
determining the first cardiovascular score based on output of the convolutional block and output of the subject demographic block.

15. The method of claim 1, wherein determining the first cardiovascular score using the first trained AI model includes:
providing the plurality of ECG data windows as input to a convolutional block of the first trained AI model;
providing the one or more demographics as input to a subject demographic block of the first trained AI model;
providing output of the convolutional block and output of the subject demographic block as input to one or more fully connected layers; and
determining the first cardiovascular score based on output of the one or more fully connected layers.

16. The method of claim 1, wherein providing the notification based on the indication of the cardiovascular condition includes:
providing the notification recommending that the subject be scheduled for a diagnostic test to confirm the indication of the cardiovascular condition.

17. A system comprising:
one or more processors; and
one or more non-transitory computer-readable media storing contents executable by the one or more processors to cause the system to:
receive electrocardiogram (ECG) data of a subject, wherein the ECG data includes a time series of voltage measurements;
determine a plurality of ECG data windows based on ECG data;
obtain one or more demographics of the subject;
provide the plurality of ECG data windows and the one or more subject demographics as input to an ensemble of trained AI models, wherein the ensemble of trained AI models includes AI models having at least two different AI model frameworks;
determine a plurality of cardiovascular scores using the ensemble of trained AI models;
determine a collective cardiovascular score based on the plurality of cardiovascular scores;
obtain an operating point threshold based on the one or more demographics;
compare the collective cardiovascular score to the operating point threshold; and
determine an indication of a cardiovascular condition based on the comparison.

18. The system of claim 17, wherein the one or more processors provide the subject demographics as input to the plurality of trained AI models by being further configured to:
provide the subject demographics to a subject demographic block of each trained AI model of the ensemble of trained AI models.

19. One or more non-transitory computer-readable storage media collectively storing contents executable by one or more processors to perform actions, the actions comprising:
obtaining a plurality of ECG data windows based on ECG data of a subject;
obtaining one or more demographics of the subject;
providing the plurality of ECG data windows and the one or more demographics as input to a first trained artificial intelligence (AI) model having a first framework and to a second trained AI model having a second framework different from the first framework;
determining, using the first trained AI model, a first pulmonary score;
determining, using the second trained AI model, a second pulmonary score;
determining an overall pulmonary score based on the first pulmonary score and the second pulmonary score;
obtaining an operating point threshold based on the one or more demographics;
comparing the overall pulmonary score to the operating point threshold; and
determining an indication of a pulmonary condition based on the comparison.

20. The one or more non-transitory computer-readable storage media of claim 19, wherein obtaining the plurality of ECG data windows based on the ECG data includes:
segmenting the ECG data into the plurality ECG data windows, wherein each ECG data window overlaps with at least one other ECG data window.
